(19) United States
(12) Patent Application Publication
(10) Pub. No.: US 2026/0083662 A1
(43) Pub. Date: Mar. 26, 2026

(54) SKINCARE AND THERAPEUTIC BALM AND PREPARATION METHOD THEREOF

(71) Applicant: Fine Washed LLC, West New York, NJ (US)

(72) Inventor: Dounya Mohamed Yahiaoui, Wayne, NJ (US)

(73) Assignee: Fine Washed LLC, West New York, NJ (US)

(21) Appl. No.: 18/890,852

(22) Filed: Sep. 20, 2024

(51) Int. Cl. A61K 8/92; A61K 8/9789; A61Q 19/00

(52) U.S. Cl. CPC A61K 8/925; A61K 8/927; A61K 8/9789; A61Q 19/00; A61Q 19/001

(57) ABSTRACT

A high-quality skincare and therapeutic balm, and preparation method therefor. The skincare and therapeutic balm consists of an Ethiopian sourced black seed oil combined with Illinois sourced beef tallow, and is created by heating and mixing a predetermined amount of beef tallow until a white creamy consistency is obtained, and then pouring a predetermined amount of black seed oil into the white creamy beef tallow mixture while continuously whisking to obtain an enhanced balm product consistency. The resulting skincare and therapeutic balm provides an effective skin care and therapeutic benefits.

### STATEMENT REGARDING FEDERALLY SPONSORED RESEARCH OR DEVELOPMENT

Not applicable.

### COPYRIGHT NOTICE

A portion of this disclosure contains material which is subject to copyright protection. The copyright owner has no objection to the photocopy reproduction by anyone of the patent document or the patent disclosure in exactly the form it appears in the Patent and Trademark Office patent file or records, but otherwise reserves all copyright rights whatsoever. 37 C.F.R 1.71(d).

### FIELD OF THE INVENTIVE CONCEPT

The present inventive concept relates to a skincare and therapeutic balm, and preparation method thereof. More particularly, but not exclusively, the present inventive concept relates to a skincare and therapeutic balm having an enhanced texture and efficacy, and preparation method thereof.

### BACKGROUND OF THE INVENTIVE CONCEPT

Skincare and therapeutic balms are commonly used for purposes of avoiding dry peeling skin and for healing dried skin and keeping skin moist over long periods of time, as well as for providing therapeutic effects on the skin and underlying nerves. However, many balms for skincare and therapeutic purposes remain oily on the skin or lose their effect on the skin quickly. Historically people used herbal remedies for medicinal purposes, as well as to cure infections and even diseases, as well as other purposes.

### SUMMARY OF THE INVENTIVE CONCEPT

The present general inventive concept is directed to a skincare and therapeutic balm that prevents drying and peeling of the skin, provides therapeutic benefits, includes anti-inflammatory and antioxidant properties, and has an enhanced texture and efficacy, and preparation method thereof.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a skincare and therapeutic balm, consisting of the following components: 7 pounds (lbs) of whisked creamy beef tallow mixed with two tablespoons of black seed oil to result in a balm consistency to be applied to and absorbed by a human's skin.

In an example embodiment, the black seed oil is an Ethiopian Black Seed Oil sourced from Ethiopia.

In an example embodiment, the beef tallow is sourced from beef raised in Illinois.

The foregoing and/or other features and utilities of the present general inventive concept may also be achieved by providing a method of forming a skincare and therapeutic balm, comprising the following steps: heating 3.5 pounds of beef tallow to a melting point to reach a liquid consistency; adding the 3.5 pounds of melted beef tallow to 3.5 pounds of room temperature beef tallow; mixing the 3.5 pounds of melted beef tallow and the 3.5 pounds of room temperature beef tallow until the mixture becomes very white and creamy; pouring three tablespoons (or 44.4 milliliters) or black seed oil into the white and creamy mixture of beef tallow while continuously whisking the mixture; perform homogenization of the mixture of beef tallow and black seed oil until a balm consistency results; and pouring the homogenized mixture into cobalt blue jars and result the mixture in the jars for at least two hours.

In an example embodiment, the black seed oil is sourced from Ethiopia.

In another example embodiment, the beef tallow is an Illinois sourced beef tallow.

Additional features and utilities of the present general inventive concept will be set forth in part in the description which follows and, in part, will be obvious from the description, or may be learned by practice of the general inventive concept.

The foregoing and/or other features and utilities of the present general inventive concept may also be achieved by providing a beef tallow and black seed oil lip balm, consisting of the following components: 8 pounds of beef tallow; 2-3 ounces of beeswax; and 1 teaspoon of black seed oil.

In an example embodiment the black seed oil is an Ethiopian Black Seed Oil sourced from Ethiopia.

In another example embodiment the beef tallow is sourced from beef raised in Illinois.

The foregoing and/or other features and utilities of the present general inventive concept may also be achieved by providing a method of making a beef tallow and black seed oil lip balm, the method comprising the following steps: heating 4 pounds of beef tallow with 2-3 ounces of beeswax in a heat-resistant bowl until the mixture is fully melted; remove the melted mixture of 4 pounds of beef tallow and 2-3 ounces of beeswax from heat and stirring in 1 tablespoon of black seed oil to the mixture until fully incorporated; adding 4 more pounds of beef tallow to the mixture and stirring continuously until fully integrated and a smooth uniform consistency is achieved; and pouring the fully integrated mixture into lip balm sized containers and letting cool to room temperature until solidified.

In an example embodiment the black seed oil is Ethiopian sourced black seed oil.

In another example embodiment the beef tallow is sourced from beef raised in Illinois.

### BRIEF DESCRIPTION OF THE DRAWINGS

These and/or other features and utilities of the present inventive concept will become apparent and more readily appreciated from the following description of the embodiments, taken in conjunction with the accompanying drawings of which:

FIG. 1 illustrates a process for preparing a skincare and therapeutic balm, according to an example embodiment of the present inventive concept.

FIG. 2 illustrates a process for making a halal beef tallow and black seed oil lip balm, according to another example embodiment of the present inventive concept.

The drawing illustrates a process for making the invention according to an example embodiment of the present inventive concept and is not to be considered limiting in its scope, as the overall inventive concept may admit to other equally effective embodiments.

### DETAILED DESCRIPTION OF THE PREFERRED EMBODIMENTS

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and is made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself Hereinafter, one or more exemplary embodiments of the present general inventive concept will be described in detail with reference to accompanying drawings.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein as understood by the ordinary artisan based on the contextual use of such term differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawing and described below.

The present disclosure includes many aspects and features. Moreover, while many aspects and features relate to, and are described in the context of methods to prepare a therapeutic balm, embodiments of the present disclosure are not limited to use only in this context.

### Skin Care and Therapeutic Balm

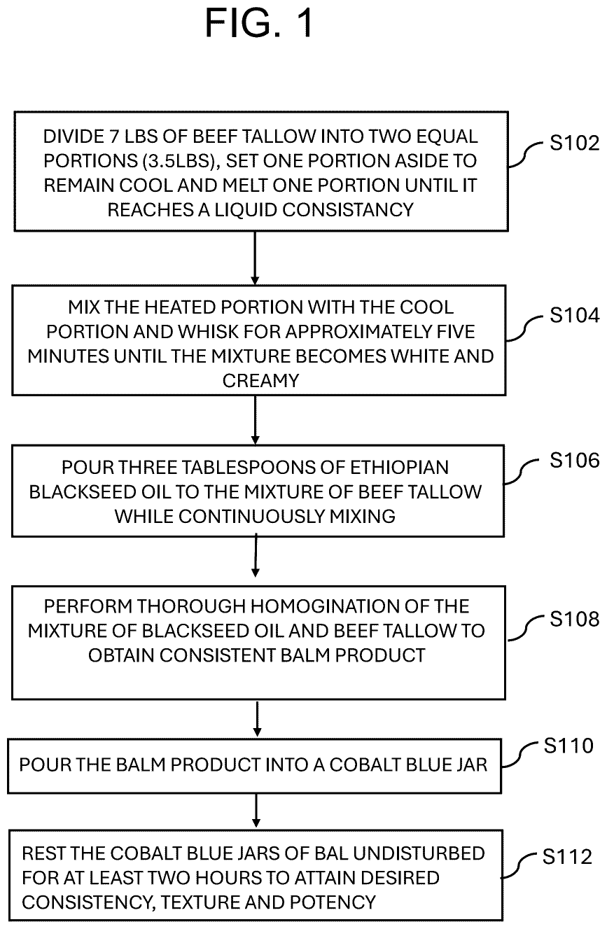

FIG. 1 illustrates a process for making a skincare and therapeutic balm, according to an example embodiment of the present inventive concept. At Step S102, measure out 7 pounds (lbs) of beef tallow (preferably halal beef tallow) and divide into two equal 3.5 lb portions. Place one 3.5 lb portion aside and keep cool while heating the other 3.5 lb portion until it becomes melted and reaches a liquid consistency. This process takes approximately fifteen minutes. In Step S104 the melted 3.5 lb portion of the halal beef tallow is mixed into the cool 3.5 lb portion of the halal beef tallow, preferably with a hand whisk, for approximately five minutes until the entire 7 lb mixture becomes very white and creamy. It is to be noted that hand whisking is preferred over an electrical mixture to obtain the best results.

At Step S106, after the 7 lb mixture of halal beef tallow becomes white and creamy, add 3 tablespoons (approximately 44.4 milliliters (ml)) of blackseed oil, preferably from Ethiopia to the halal beef tallow mixture. According to an example embodiment, the mixture of blackseed oil and beef tallow should undergo a thorough homogenization process at Step S108 in order to ensure uniform distribution of the black seed oil within the beef tallow base, resulting in a consistent balm product. Then at Step S110 the balm is poured into two or four ounce cobalt blue jars, which are specifically chosen for their protective properties against light degradation. Then at Step S112 the filled cobalt blue jars rest undisturbed for a minimum of two hours, during which time the balm matures to attain a desired consistency, texture and potency. The synergistic blend of the black seed oil and beef tallow imbues the balm with potent therapeutic properties, providing relief from various skin ailments and promoting an overall well-being. The cobalt blue jars are not only important for protecting the resulting balm product from light-inducted degradation, but also for preserving the balm's integrity and efficacy over a long period of time, as well as for providing an aesthetic appeal to the consumer.

In an example embodiment the black seed oil is an Ethiopian Black Seed Oil, sourced from Ethiopia. Ethiopian Black Seed Oil is known for its numerous health benefits, including anti-inflammatory and antioxidant properties. In another example embodiment the beef tallow is specifically sourced from Illinois. Illinois sourced beef tallow is known to be high-quality beef tallow, which provides a rich and nourishing base for the balm, as well as enhancing the texture and efficacy of the balm. The resulting product according to this example embodiment is a halal tallow black seed oil balm, which uses halal-certified ingredients and manufacturing processes to ensure compliance with Islamic dietary laws, and also provides inclusion for a broader consumer base.

### Lip Balm

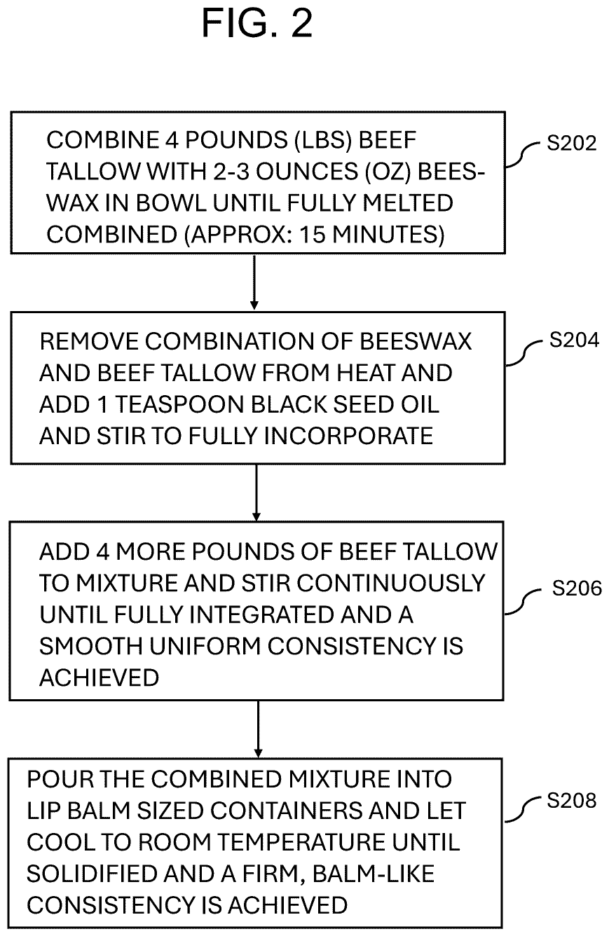

FIG. 2 illustrates a process for making a halal beef tallow and black seed oil lip balm, according to another example embodiment of the present inventive concept. At Step S202 four pounds (lbs) of beef tallow are combined with approximately two-three ounces (oz) of beeswax and heated together in a heat-resistant bowl until the mixture of beef tallow and beeswax are fully melted and combined, which will take approximately fifteen minutes. The combined mixture should become smoothly blended and uniform.

At Step S204 the melted combination of beef tallow and beeswax should be removed from the heat, at which point one teaspoon of black seed oil is to be added to the melted mixture. The combination of melted beef tallow, beeswax and black seed oil should be stirred until the black seed oil is fully incorporated into the mixture of beef tallow and beeswax.

At Step S206 four more pounds of beef tallow should be added to the fully combined mixture of beef tallow, beeswax and black seed oil until the additional four pounds of beef tallow is fully integrated into the mixture such that a smooth uniform consistency is achieved.

Finally, at Step S208 the fully integrated mixture should be poured into lip balm sized containers and let cool at room temperature until solidified to achieve a firm, balm-like consistency suitable for application to lips.

In an example embodiment the black seed oil is an Ethiopian Black Seed Oil, sourced from Ethiopia. Ethiopian Black Seed Oil is known for its numerous health benefits, including anti-inflammatory and antioxidant properties. In another example embodiment the beef tallow is specifically sourced from Illinois.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A skincare and therapeutic balm, consisting of the following components:
7 pounds of whisked creamy beef tallow; and
2 tablespoons of black seed oil.

2. The skincare and therapeutic balm according to claim 1, wherein the black seed oil is an Ethiopian Black Seed Oil sourced from Ethiopia.

3. The skincare and therapeutic balm according to claim 2, wherein the beef tallow is sourced from beef raised in Illinois.

4. A method of making a skincare and therapeutic balm, the method comprising the following steps:
heating 3.5 pounds of beef tallow to a melting point to reach a liquid consistency;
adding the 3.5 pounds of melted beef tallow to 3.5 pounds of room temperature beef tallow;
mixing the 3.5 pounds of melted beef tallow and the 3.5 pounds of room temperature beef tallow until the mixture becomes very white and creamy;
pouring 3 tablespoons (or 44.4 milliliters) or black seed oil into the white and creamy mixture of beef tallow while continuously whisking the mixture;
perform homogenization of the mixture of beef tallow and black seed oil until a balm consistency results;
pour the homogenized mixture into cobalt blue jars and result the mixture in the jars for at least two hours.

5. The method according to claim 4, wherein the black seed oil is Ethiopian sourced black seed oil.

6. The method according to claim 5, wherein the beef tallow is Illinois sourced beef tallow.

7. A beef tallow and black seed oil lip balm, consisting of the following components:
8 pounds of beef tallow;
2-3 ounces of beeswax; and
1 teaspoon of black seed oil.

8. The skincare and therapeutic balm according to claim 7, wherein the black seed oil is an Ethiopian Black Seed Oil sourced from Ethiopia.

9. The skincare and therapeutic balm according to claim 8, wherein the beef tallow is sourced from beef raised in Illinois.

10. A method of making a beef tallow and black seed oil lip balm, the method comprising the following steps:
heating 4 pounds of beef tallow with 2-3 ounces of beeswax in a heat-resistant bowl until the mixture is fully melted;
remove the melted mixture of 4 pounds of beef tallow and 2-3 ounces of beeswax from heat and stirring in 1 tablespoon of black seed oil to the mixture until fully incorporated;
adding 4 more pounds of beef tallow to the mixture and stirring continuously until fully integrated and a smooth uniform consistency is achieved; and
pouring the fully integrated mixture into lip balm sized containers and letting cool to room temperature until solidified.

11. The method according to claim 10, wherein the black seed oil is Ethiopian sourced black seed oil.

12. The method according to claim 11, wherein the beef tallow is Illinois sourced beef tallow.
